(19) United States
(12) Patent Application Publication
(10) Pub. No.: US 2019/0385732 A1
(43) Pub. Date: Dec. 19, 2019

(54) MEDICAL TOOLS MANAGEMENT SYSTEM AND METHODS FOR OPERATING SAME

(71) Applicant: SURGICAL SAFETY SYSTEMS, LLC, Brighton, MI (US)

(72) Inventors: Fredrick P. Schoville, Brighton, MI (US); John Richard Dein, Fair Oaks, CA (US)

(21) Appl. No.: 16/463,871

(22) PCT Filed: Nov. 28, 2017
(86) PCT No.: PCT/US17/63538

§ 371 Date: May 24, 2019

Related U.S. Application Data
(60) Provisional application No. 62/426,845, filed on Nov. 28, 2016.

(51) Int. Cl. G16H 40/20; G16H 40/40

(52) U.S. Cl. CPC G16H 40/20; A61B 90/90; G16H 40/40

(57) ABSTRACT

Method and system is disclosed for tracking and managing medical tools associated with a medical procedure. The method includes receiving a list of medical tools or instruments, associating each medical tool within the list a status of either checked-in status or checked-out status, wherein the status is defaulted to the checked-out status, monitoring one of: a weight measurement, and information associated with a radio frequency identification (RFID), wherein the weight measurement is associated with a weight of objects placed on a defined surface area, detecting a presence of a first medical tool within a zone proximate to the defined surface area, and changing the status associated with the first medical tool to checked-in status based upon the detecting.

### CROSS REFERENCE TO RELATED APPLICATIONS

This application claims the benefit of Provisional Application No. 62/426,845 filed on Nov. 28, 2016 which is hereby incorporated herein by reference.

### TECHNICAL FIELD

This disclosure relates to medical tool preparation and management, and more particularly to systems and methods for tracking and managing medical tools associated with a medical procedure.

### BACKGROUND

The statements in this section merely provide background information related to the present disclosure and may not constitute prior art.

Presently in the surgical suite instruments are manually counted by an operating room technician and a nurse to confirm that the medical instruments are present at the beginning of a medical procedure. Counting is generally done before the medical procedure, i.e., “pre-op” and at the end of the surgery, i.e., “post-op.” While the instruments are in use, it can be difficult to track and manage the medical instruments manually, particularly during long and complex procedures.

Therefore, a need exists for a system to wirelessly manage the medical tools, including a quantity and a tool location, before, during, and after the medical procedure.

### SUMMARY

Method and system is disclosed for tracking and managing medical tools associated with a medical procedure. The method includes receiving a list of medical tools or instruments, associating each medical tool within the list a status of either checked-in status or checked-out status, wherein the status is defaulted to the checked-out status, monitoring one of: a weight measurement, and information associated with a radio frequency identification (RFID), wherein the weight measurement is associated with a weight of objects placed on a defined surface area, detecting a presence of a first medical tool within a zone proximate to the defined surface area, and changing the status associated with the first medical tool to checked-in status based upon the detecting.

This summary is provided merely to introduce certain concepts and not to identify key or essential features of the claimed subject matter.

### BRIEF DESCRIPTION OF THE DRAWINGS

One or more embodiments will now be described, by way of example, with reference to the accompanying drawings, in which:

FIG. 1 schematically shows an exemplary medical tool management system, in accordance with the present disclosure;

FIG. 2A shows an exemplary scanner device, in accordance with the present disclosure;

FIG. 2B shows an exemplary underside of an embodiment of the scanner device, in accordance with the present disclosure;

FIG. 2C shows an exemplary table surface 14 for illustrating signal receiving zones, in accordance with the present disclosure;

FIG. 3 schematically shows the exemplary scanner device, in accordance with the present disclosure; and

FIG. 4 is a logical flow diagram illustrating one embodiment of the generalized method for tracking and managing medical tools associated with a medical procedure, in accordance with the present disclosure.

### DETAILED DESCRIPTION

Reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment.

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” The term “based upon” is not exclusive and allows for being based on additional factors not described, unless the context clearly dictates otherwise. Additionally, in the subject description, the word “exemplary” is used to mean serving as an example, instance or illustration. Any embodiment or design described herein as “exemplary” is not necessarily to be construed as preferred or advantageous over other embodiments or designs. Rather, use of the word exemplary is intended to present concepts in a concrete manner.

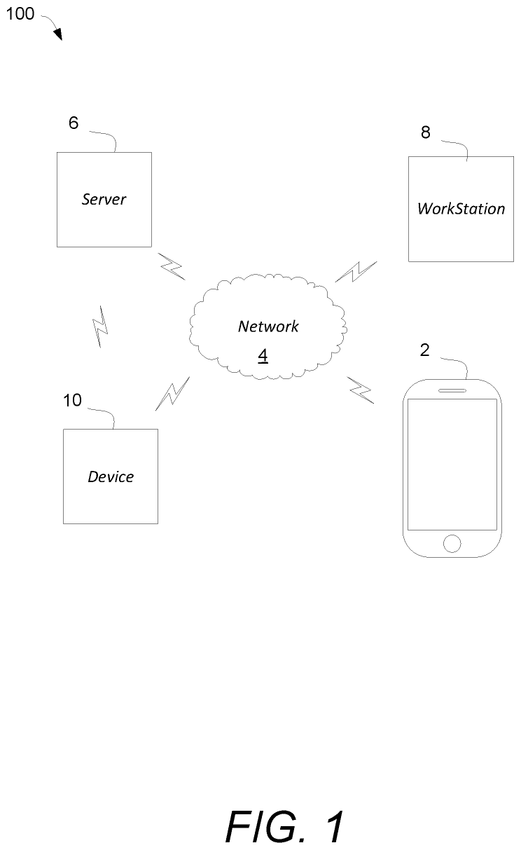

Referring now to the drawings, wherein the depictions are for the purpose of illustrating certain exemplary embodiments only and not for the purpose of limiting the same, FIG. 1 schematically shows an exemplary medical tool management system 100 that may help implement the methodologies of the present disclosure. The system 100 can include a mobile device 2, a network 4, a server system 6, a workstation computer 8, and a medical tool scanner device 10. As shown in FIG. 1, the scanner device 10 may be communicatively connected to one or more computing devices including a server 6, a workstation computer 8, and/or a mobile device 2. The scanner device 10 may be indirectly communicatively connected to the one or more computing devices via the network 4 or directly communicatively connected via wireless protocol. Any one or more of the one or more computing devices may be communicatively connected to one another. For example, the server system 6 may be directly communicatively connected to the workstation computer 8 and the mobile device 2. The mobile device 2 may be physically connected to the network 4 or the workstation computer 8 during selected periods of operation without departing from the teachings herein. Components of the system 100 are shown in FIG. 1 as single elements. Such illustration is for ease of description and it should be recognized that the system 100 may include multiple additional mobile and computing devices. In various embodiments, the system 100 may operate without one or more of the mobile device 2, the network 4, the server system 6, and the workstation computer 8.

The network 4 may be any suitable series of points or nodes interconnected by communication paths such as a local wired and/or wireless network. The network 4 may be interconnected with other networks and contain sub networks network such as, for example, a publicly accessible distributed network like the Internet or other telecommunications networks (e.g., intranets, virtual nets, overlay networks and the like). The network 4 may facilitates the exchange of data between and among the scanner device 10, the mobile device 2, the workstation computer 8, and the server system 6, as requested.

The server system 6 and/or the workstation computer 8 may each be: various embodiments of a computer including high-speed microcomputers, minicomputers, mainframes, and/or data storage devices. The server system 6 preferably executes database functions including storing and maintaining a database and processes requests from the scanner device 10, the mobile device 2 and/or the workstation computer 8 to extract data from, or update, a database as described herein below. The server 6 may additionally provide processing functions for the scanner device 10, the mobile device 2 and the workstation computer 8 as will become apparent to those skilled in the art upon a careful reading of the teachings herein.

In addition, one or more of the scanner device 10, the mobile device 2 and the workstation computer 8 may include one or more applications that the user may operate. Operation may include downloading, installing, turning on, unlocking, activating, or otherwise using the application. The application may comprise at least one of an algorithm, software, computer code, and/or the like, for example, mobile application software. In the alternative, the application may be a website accessible through the world wide web, for example.

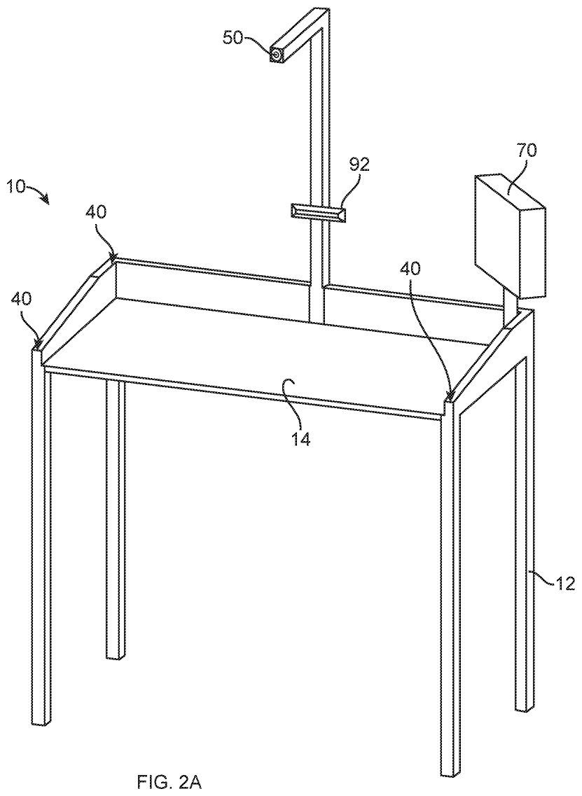

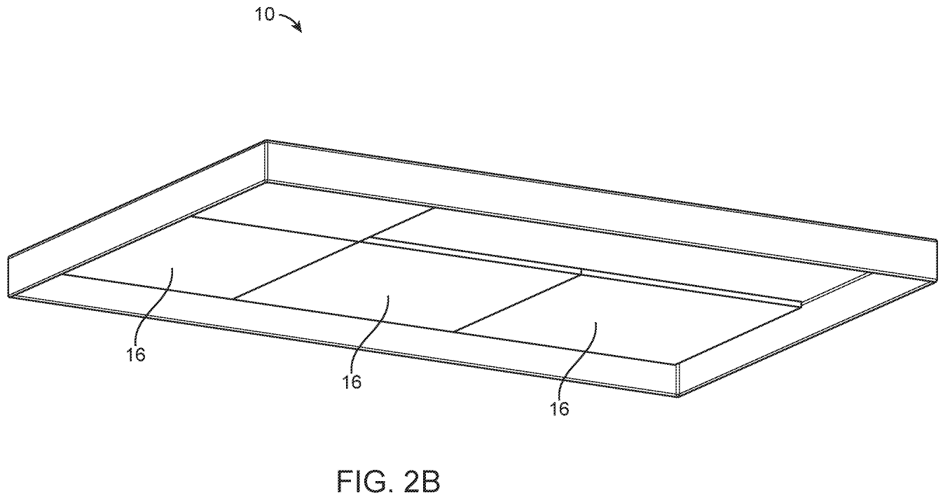

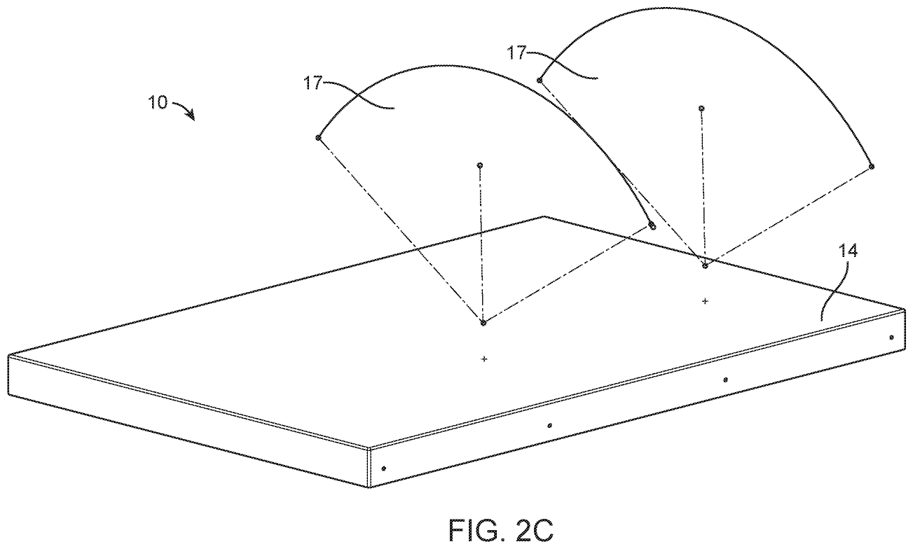

FIG. 2A schematically shows an embodiment of the scanner device 10. As FIG. 2 shows, the scanner device 10 may be elevated by legs 12. In other embodiments, the scanner device 10 may be elevated a cabinet or shelf, for example. In one embodiment, the scanner device 10 includes a camera 50. In one embodiment, the scanner device 10 includes a weight scale built into a surface 14. The weight scale may be configured to weigh an object that rests on the surface 14. The scanner device 10 may include one or more radio-frequency identification (RFID) sensors 40, each proximate to a corner, in one embodiment.

In one embodiment, the scanner device 10 includes a medical instrument present sensor 92. This sensor 92 may be implemented as one or more of a motion detector, a passive infrared sensor, a microwave-based transceiver, an ultrasonic transceiver, a tomographic motion detector, and a digital camera-based sensor.

The scanner device 10 preferably includes a computer module 70 configured to power and enable various components of the device 10. For example, the weight scale is preferably communicatively connected to the computer module 70 so that the module 70 may monitor and communicate weight measurements as desired or requested according to methods disclosed herein. Similarly, the RFID sensor(s) 40, the camera 50 and/or the sensor 92 may be communicatively connected to the computer module 70. The computer module 70 includes a display monitor in one embodiment.

FIG. 2B shows an underside 15 of the surface 14 of an embodiment of the scanner device 10. The underside 15 may include a plurality of antenna tiles 16. The antenna tiles 16 may supplement the RFID sensor(s) 40 or be used in place of them. In one embodiment, each of the antenna tiles 16 include one or more antennas configured to receive RFID signals for transmission to the RFID reader 40.

FIG. 2C shows an exemplary table surface 14. Signal receiving zones 17 may extend to the table surface 14. The signal receiving zones 17 may be defined as the space proximate to the surface 14 that is able to receive RFID signals.

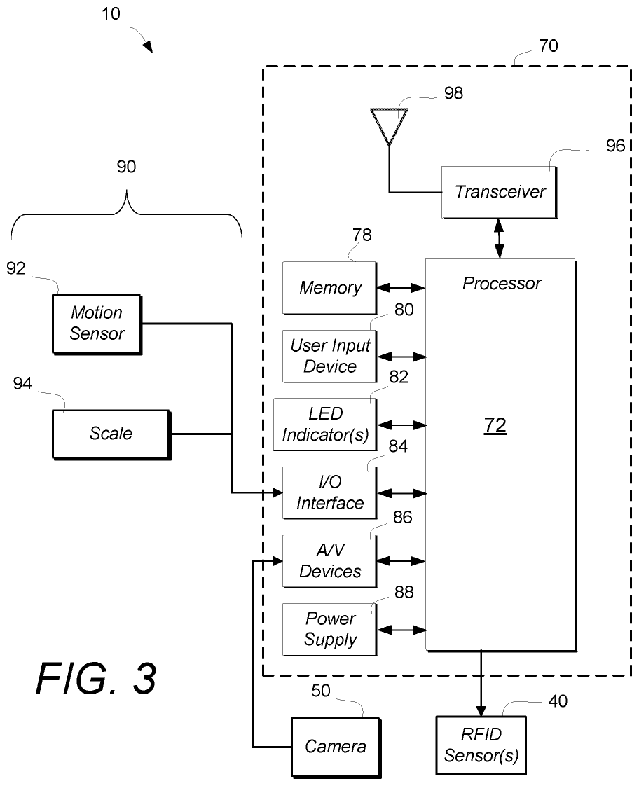

FIG. 3 schematically shows an exemplary embodiment of the scanner device 10. As shown in FIG. 3, the device 10 includes a processor module 72. The device 10 may additionally include any digital and/or analog circuit elements, comprising discrete and/or solid state components, suitable for use with the embodiments disclosed herein. One skilled in the art will recognize upon a careful reading of the teachings herein that a radio processor may be included in another embodiment of the device 10. In one embodiment, a communication adapter and/or transceiver is utilized for wireless communication over one or more wireless communications channels. Although various components are shown as separate components, such an illustration is for ease of description and it should be recognized that the functions performed by the various components may be combined on one or more components.

The processor module 72 may be configured to execute various computer programs (e.g., software, firmware, or other code) such as application programs and system programs to provide computing and processing operations for the device 10. In various embodiments, processor module 72 may be implemented as a host central processing unit (“CPU”) using any suitable processor or logic device, such as a general purpose processor, or other processing device in alternative embodiments configured to provide processing or computing resources to device 10. For example, processor module 72 may be responsible for executing various computer programs such as application programs and system programs to provide computing and processing operations for device 10. The application software may provide a graphical user interface (“GUI”) to communicate information between device 10 and a user. The computer programs may be stored as firmware on a memory associated with processor 72, may be loaded by a manufacturer during a process of manufacturing device 10, and may be updated from time to time with new versions or software updates via wired or wireless communication.

System programs assist in the running of a computer system. System programs may be directly responsible for controlling, integrating, and managing the individual hardware components of the computer system. Examples of system programs may include, for example, an operating system, a kernel, device drivers, programming tools, utility programs, software libraries, an application programming interface (“API”), a GUI, and so forth.

The memory module 78 is preferably coupled to the processor module 72. In various embodiments, the memory module 78 may be configured to store one or more computer programs to be executed by the processor module 72. The memory module 78 may be implemented using any machine-readable or computer-readable media capable of storing data such as volatile memory or non-volatile memory, removable or non-removable memory, erasable or non-erasable memory, writeable or re-writeable memory, and so forth. Although the memory module 78 is shown as being separate from the processor module 72 for purposes of illustration, in various embodiments some portion or the entire memory module 78 may be included on the same integrated circuit as the processor module 72. Alternatively, some portion or the entire memory module 78 may be disposed on an integrated circuit or other medium (e.g., solid state drive) external to the integrated circuit of the processor module 72.

A user input device 80 may be coupled to the processor module 72. The user input device 80 may include, for example, an alphanumeric, numeric key layout and an integrated number dial pad. The device 10 also may include various keys, buttons, and switches such as, for example, input keys, preset and programmable hot keys, left and right action buttons, a navigation button such as a multidirectional navigation button, power/end buttons, preset and programmable shortcut buttons, a volume control switch, a keypad and so forth. In one embodiment, the device 10 simply includes an ON and an OFF button, the other controls being activated through a wirelessly connected computing device, such as the workstation 8.

The processor module 72 may be coupled to one or more light-emitting diodes (LEDs) 82. In one embodiment, a first LED of the one or more LEDs is used to indicate a first status. In one embodiment, a second LED is used to indicate a second status. In one embodiment, the first status is associated with a green color and the second status is associated with a red color. In one embodiment, a third LED may be used to associate with a third status, e.g., a yellow color.

An I/O interface 84 is preferably coupled to the processor module 72. The I/O interface 84 may include one or more I/O devices such as a serial connection port, an infrared port, wireless capability, and/or integrated 802.11x (WiFi) wireless capability, to enable wired (e.g., USB cable) and/or wireless connection to a local or networked computer system, such as the workstation 8, and/or the server 6.

In one embodiment, the device 10 includes an audio/video (“A/V”) module 86 coupled to the processor module 72 for communicatively connecting and communicating therebetween to various audio/video devices. The A/V module 86 may be configured to support A/V capability of the device 10 including components such as, a microphone, one or more speakers, an audio port to connect an audio headset, an audio coder/decoder (codec), an audio player, a video codec, a video player, and so forth. The A/V input module 86 may include an imaging module configured to capture digital images. The imagining module may include an optical sensor, e.g., a charged coupled device (CCD) or a complementary metal-oxide semiconductor (CMOS) optical sensor to facilitate camera functions, such as recording photographs and video clips. The image frames may be processed by the memory 78 or displayed on the display 82.

A power supply 88 configured to supply and manage power to components of device 10 is preferably coupled to the processor module 72. In various exemplary embodiments, the power supply 88 may be implemented by a rechargeable battery, such as a removable and rechargeable lithium ion battery to provide direct current (“DC”) power, and/or an alternating current (“AC”) adapter to draw power from a standard AC main power supply.

The device 10 may include one or more transceivers 96 coupled to the processor 72 and an antenna 98, each transceiver may be configured to communicate using different types of protocol, e.g., Bluetooth®, Near Field Communications, Mesh network, etc., communication ranges, operating power requirements, RF sub-bands, information types (e.g., voice or data), use scenarios, applications, and so forth. For example, the transceiver 96 may include a Wi-Fi transceiver and a cellular or WAN transceiver configured to operate simultaneously. In various embodiments, the transceiver is alternated for a transmitter and/or receiver.

The radio-frequency identification (RFID) sensor(s) 40 may be coupled to the host processor 72. In one embodiment, functions of the RFID reader 40 are incorporated into the processor 72 enabling the device 10 to utilizes the transceiver 96 and antenna 98 to carry out functions of the RFID reader 40. The RFID reader 40 is configured to read identification information stored on an RFID tag by use of radio waves. In one embodiment, an RFID tag is formed of a microchip that is attached to an antenna and upon which is stored a unique digital identification number.

In one embodiment, the device 10 includes a plurality of sensors 90. The sensors may be directly coupled to the processor 72 or connected through one or more other modules including, e.g., the I/O interface, such as shown in FIG. 3. In one embodiment, a motion sensor 92 is included. In one embodiment, a weight scale sensor 94 is included.

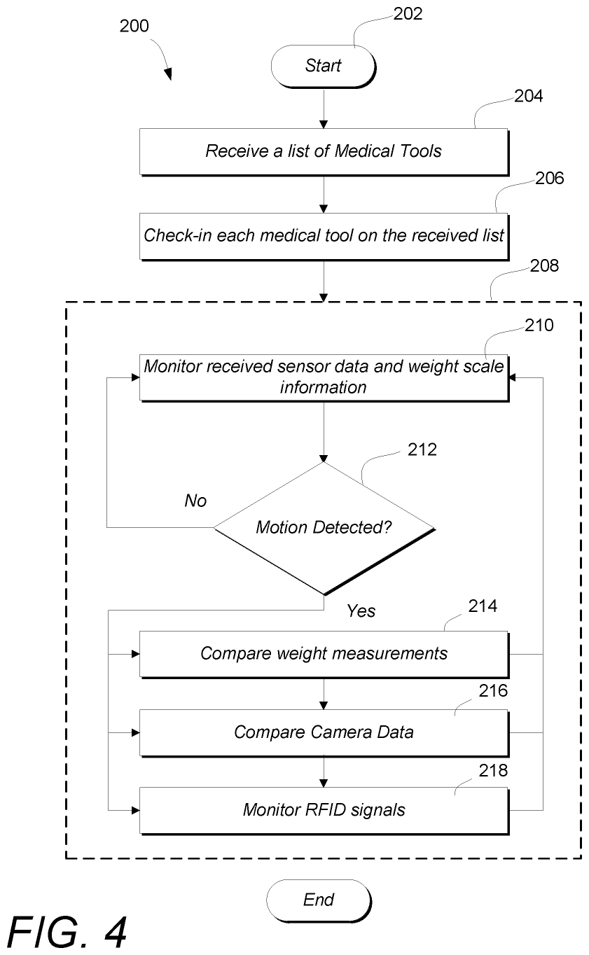

Referring now to FIG. 4, one embodiment of a generalized method 200 for tracking and managing medical tools associated with a medical procedure is illustrated. The method 200 may be utilized in conjunction with the system 100 and the device 10. The device 10 is capable of selectively communicating with one or more computing devices within the exemplary system 100 as will be discussed in more detail below.

The method 200 may be initialized manually or automatically in accordance with other executing processes. In one embodiment, the method 200 is initialized by simply turning the device 10 to an ON operating state. In one embodiment, the method 200 is initialized by receiving instructions from a computer program or operator to start 202.

At step 204, the device 10 receives information associated with a set of medical tools and instruments associated with an upcoming medical procedure. In one embodiment, the information includes a list of medical tools necessary for the procedure and a check-in status of each item contained within the list.

At step 206, each medical tool is checked in. The check in procedure may include individually placing each medical tool on the surface 14 of the device 10. The device 10 may establish a positive check-in status by confirming the identification of the tool via an RFID tag, weight measurement, and/or graphical camera data. The instrument present sensor 92 may be utilized to begin or end identification of the medial tool. For example, initially the surface 14 is clear of medical tools. Upon breaking a zone proximate to the surface, the instrument present sensor 92 reports that a medical tool is present. This reporting may be used to initialize further sensor inquiry into the identification of the medical tool. For example, upon reporting that a medical tool is present, the RFID sensor 40 may turn to an ON operating status and attempt to read an RFID tag.

At step 208, the check in procedure is completed and the scanner device 10 is transitioned to a real-time scanning mode of operation. Initially, all of the checked-in medical tools are initially present on the scanner device 10.

During the real-time scanning mode, the scanner device 10 monitors information from the sensors 40 and 92, the camera 50, and the weight scale 94 at step 210. In one embodiment, the scanner device 10 waits until the motion detector sensor 92 indicates that a user's hand is within a zone proximate to the surface 14, i.e., a detection zone is breached, at step 212. This indication may be made in any number of ways depending upon the specific type of motion detector is utilized.

After a breach is detected, the scanner device 10 attempts to monitor and determine what medical instruments are being removed or returned on the surface 14. In one embodiment, the scanner device 10 will monitor for discrete changes in weight measurements. Discrete changes in weight measurements can be used to determine a weight of an item that has been removed or returned 214. For example, an initial weight may be compared with a weight measurement after a weight change has occurred. The difference between the measurements may indicate loss or gain of a medical tool. Further, the specific difference may indicate a specific tool that was removed or returned to the surface 14.

At Step 216, the scanner device 10 may use camera data to execute object recognition algorithms to determine removal or placement of a medical tool on the surface 14. In one embodiment, breach of the detection zone may initialize the camera to record an image or record a video. In this way, a visual record of the change in medical tools may be obtained for subsequent retrieval. In one embodiment, the camera data may be associated with a particular medical tool if the medical tool had a status change, i.e., from checked-in to checked-out, or vice versa.

At step 218 the scanner device 10 may monitor and detect RFID tags from one or more medical tools. In one embodiment, signal attenuation or amplification associated with a particular signal is indicative of a removal or return of a medical tool. In one embodiment, signals from the one or more RFID sensors 40 may be compared to determine whether an RFID tag on a medical device is moving, which is indicative of its removal or return. In one embodiment, a sensor zone is established over the surface 14 in a way that does not detect RFID tags on the medical instruments on the surface 14, but detects RFID tags as they are placed on the surface 14 or removed therefrom. In one embodiment, camera data supplements RFID sensor data to determine if the medical tool was removed or returned. For example, an object, post detection of a change in RFID signals, being detected by the camera indicates the return of the item, while an absence of an object from the surface 14 indicates the removal of the item.

In one embodiment, location of the medical tools may be tracked. Location may be determined based upon GPS data and/or a module from iBeacon\hat{ }™. For example, tools associated with a location having a distance greater than a predefined distance from the device 10 may be determined to be checked-out, while tools within the predefined proximity may be determined to be checked-in.

The schematic flow chart diagrams included herein are generally set forth as logical flow chart diagrams. As such, the depicted order and labeled steps are indicative of one embodiment of the presented process. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the process. For example, an arrow may indicate a waiting or monitoring period of unspecified duration between enumerated steps of the depicted process. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Additionally, the order in which a particular method occurs may or may not strictly adhere to the order of the corresponding steps shown. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures. For example, steps 206, and 208 may be executed concurrently in some embodiments.

Additionally, examples in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of computer readable program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of computer readable program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the computer readable program code may be stored and/or propagated on in one or more computer readable medium(s).

The computer readable medium may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing.

More specific examples of the computer readable medium may include but are not limited to a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. Computer readable program code embodied on a computer readable signal medium may be transmitted using any appropriate medium, including but not limited to wireless, wireline, optical fiber cable, Radio Frequency (RF), or the like, or any suitable combination of the foregoing

In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

While the foregoing disclosure discusses illustrative embodiments, it should be noted that various changes and modifications could be made herein without departing from the scope of the described embodiments as defined by the appended claims. Accordingly, the described embodiments are intended to embrace all such alterations, modifications and variations that fall within scope of the appended claims. Furthermore, although elements of the described embodiments may be described or claimed in the singular, the plural is contemplated unless limitation to the singular is explicitly stated. Additionally, all or a portion of any embodiment may be utilized with all or a portion of any other embodiments, unless stated otherwise.

1. Method for tracking and managing medical tools associated with a medical procedure, the method comprising:
receiving a list of medical tools or instruments;
associating each medical tool within the list a status of either checked-in status or checked-out status, wherein the status is defaulted to the checked-out status;
monitoring one of: a weight measurement, and information associated with a radio frequency identification (RFID), wherein the weight measurement is associated with a weight of objects placed on a surface area of a scanner device;
detecting a presence of a first medical tool within a zone proximate to the surface area; and
changing the status associated with the first medical tool to checked-in status based upon the detecting.

2. The method of claim 1, further comprising:
assigning a check-in or checked-out status based upon proximity to the surface area.

3. The method of claim 1, further comprising:
upon detection of the presence of the first medical tool with the zone, generating a video record of the first medical tool and associating the video record with the first medical tool with a database.

4. The method of claim 1, wherein the weight measurement is determined by an elevated weight measurement device.

5. The method of claim 1, further comprising:
conducting a total check-in procedure including:
weighing all medical tools on the surface area to determine a total check-out weight of all the medical tools; and
weighing all the medical tools on the surface area subsequent to a medical procedure to determine a total check-in weight of all the medical tools; and
comparing the total check-out weight to the total check-in weight.

6. The method of claim 5, further comprising:
transmitting information associated with the comparing, wherein the transmitted information includes a first information when the weight is within a predefined amount from the total check-out weight and a second information when the weight is outside a predefined amount from the total check-out weight.

7. The method of claim 5, wherein the comparing the total check-out weight to the total check-in weight is executed to determine a difference weight; and wherein the difference weight, if greater than a predefined tolerance amount, may be compared to weights associated with each medical tool on the list.

8. The method of claim 7, wherein the difference weight may be compared to weights associated with each medical tool on the list having a check-out status.

9. The method of claim 7, further comprising: determining one or more medical tools having a check-out status based upon the difference weight.

10. Method for tracking and managing medical tools associated with a medical procedure, the method comprising:
receiving a list of medical tools or instruments;
associating each medical tool within the list a status of either checked-in status or checked-out status, wherein the status is defaulted to the checked-out status;
monitoring a weight measurement, a motion detector, and information associated with a radio frequency identification (RFID), wherein the weight measurement is associated with a weight of objects placed on a surface area of a scanner device;
detecting a presence of a first medical tool within a zone proximate to the surface area, the detecting being executed based upon received information from a motion detector;
upon detecting the presence of the first medical tool by the motion detector, reading an RFID associated with the first medical tool; and
changing the status associated with the first medical tool to checked-in status based upon the reading an RFID.

11. The method of claim 10, further comprising:
assigning a check-in or checked-out status based upon proximity to the surface area.

12. The method of claim 10, further comprising:
upon detection of the presence of the first medical tool with the zone, generating a video record of the first medical tool and associating the video record with the first medical tool with a database.

13. The method of claim 10, further comprising:
conducting a total check-in procedure including:
weighing all medical tools on the surface area to determine a total check-out weight of all the medical tools; and
weighing all the medical tools on the surface area subsequent to a medical procedure to determine a total check-in weight of all the medical tools; and
comparing the total check-out weight to the total check-in weight.

14. The method of claim 13, further comprising:
transmitting information associated with the comparing, wherein the transmitted information includes a first information when the weight is within a predefined amount from the total check-out weight and a second information when the weight is outside a predefined amount from the total check-out weight.

15. The method of claim 13, wherein the comparing the total check-out weight to the total check-in weight is executed to determine a difference weight; and wherein the difference weight, if greater than a predefined tolerance amount, may be compared to weights associated with each medical tool on the list.

16. The method of claim 15, wherein the difference weight may be compared to weights associated with each medical tool on the list having a check-out status.

17. The method of claim 15, further comprising: determining one or more medical tools having a check-out status based upon the difference weight.

18. Method for tracking and managing medical tools associated with a medical procedure, the method comprising:
generating a list of medical tools or instruments based upon received radio frequency identification (RFID) information;
associating each medical tool within the list a status of either checked-in status or checked-out status, wherein the status is defaulted to the checked-out status;
monitoring a weight measurement, a motion detector, and the RFID information, wherein the weight measurement is associated with a weight of objects placed on a surface area of a scanner device;
detecting a presence of a first medical tool within a zone proximate to the surface area, the detecting being executed based upon received information from a motion detector;
upon detecting the presence of the first medical tool by the motion detector, reading an RFID associated with the first medical tool using antenna located on an underside of the surface area of the scanner device; and
changing the status associated with the first medical tool to checked-in status based upon the reading an RFID and based upon proximity to the surface area.

19. The method of claim 10, further comprising:
upon detection of the presence of the first medical tool with the zone, generating a video record of the first medical tool and associating the video record with the first medical tool with a database;
conducting a total check-in procedure including:
weighing all medical tools on the surface area to determine a total check-out weight of all the medical tools; and
weighing all the medical tools on the surface area subsequent to a medical procedure to determine a total check-in weight of all the medical tools; and
comparing the total check-out weight to the total check-in weight.

20. The method of claim 19, further comprising:
transmitting information associated with the comparing, wherein the transmitted information includes a first information when the weight is within a predefined amount from the total check-out weight and a second information when the weight is outside a predefined amount from the total check-out weight, wherein the comparing the total check-out weight to the total check-in weight is executed to determine a difference weight; and wherein the difference weight, if greater than a predefined tolerance amount, may be compared to weights associated with each medical tool on the list to identify medical tools remaining to be checked-in.
